(19) United States
(12) Patent Application Publication
(10) Pub. No.: US 2013/0267690 A1
(43) Pub. Date: Oct. 10, 2013

(54) REMOVAL OF VIRUCIDAL AGENTS FROM BIOMOLECULE PREPARATIONS

(71) Applicant: Bio-Rad Laboratories, Inc., Hercules, CA (US)

(72) Inventor: Peter S. Gagnon, Singapore (SG)

(73) Assignee: Bio-Rad Laboratories, Inc., Hercules, CA (US)

(21) Appl. No.: 13/622,093

(22) Filed: Sep. 18, 2012

Related U.S. Application Data
(60) Provisional application No. 61/536,886, filed on Sep. 20, 2011.

(51) Int. Cl. C07K 1/18

(52) U.S. Cl. CPC C07K 1/18
USPC 530/390.1; 530/387.1; 422/527

(57) ABSTRACT

Methods, compositions and kits for chromatography purification of antibodies are provided. In some embodiments, antibodies are purified by hydroxyapatite (HT) or fluorapatite (FT) that is treated with a polycationic agent. In some embodiments, the antibodies are treated with a polycationic agent that is also a virucidal agent prior to purification.

### CROSS-REFERENCE TO RELATED PATENT APPLICATIONS

The present patent application claims benefit of priority to U.S. Provisional Patent Application No. 61/536,886, filed on Sep. 20, 2011, which is incorporated by reference for all purposes.

### BACKGROUND OF THE INVENTION

The purification of biological molecules such as antibodies, other therapeutic proteins, virus and virus-like particles, and DNA plasmids for therapeutic or diagnostic purposes can be desirable. Moreover, natural and recombinant proteins produced by in vivo or in vitro methods require treatment with virucidal conditions or compounds to ensure the safety of patients receiving therapy based on those proteins. Many virucidal agents are highly toxic. A complication of virucidal treatment is that the virucidal agents themselves may form stable associations with treated protein products. These associations may make it difficult or impossible to completely remove the virucidal agent from the protein preparation.

### BRIEF SUMMARY OF THE INVENTION

The present invention provides for a two-stage (i.e., at least two-stages) viral inactivation method. In some embodiments, the method comprises incubating a biological sample comprising a target molecule with a positively-charged or neutral virucidal agent under conditions to inactivate viruses in the sample, if present; subsequently contacting the target molecule to an apatite support under conditions resulting in binding of the target biomolecule to the support such that the target biomolecule binds to the apatite and a majority of the virucidal agent flows past the support; washing the support binding the target molecule with a first wash buffer, wherein the first wash buffer comprises at least a second virucidal agent, wherein the second virucidal agent is in sufficient concentration to inactivate viruses, if present, and to dissociate complexes of the positively-charged or neutral virucidal agent and the target molecule, thereby removing at least some residual virucidal agent, if present; and eluting the target biomolecule from the support such that the target biomolecule is substantially free of the positively-charged or neutral virucidal agent.

In some embodiments, the positively-charged or neutral virucidal agent is selected the group consisting of polyethyleneimine, ethacridine, chlorhexidine, benzalkonium chloride, tri(n-butyl)phosphate, and methylene blue.

In some embodiments, the method further comprises, between the washing and eluting, contacting the support with a second wash buffer. In some embodiments, the second wash buffer has a lower conductivity than the first wash buffer and no chaotropic agents.

In some embodiments, the apatite is hydroyxapatite or fluoroapatite. In some embodiments, the apatite is in a native form at least during the contacting and washing.

In some embodiments, the apatite is in a metal-derivatized form at least during the contacting and washing. In some embodiments, the metal is a divalent or trivalent cation. In some embodiments, the metal is selected from the group consisting of calcium, iron, and zinc.

In some embodiments, the apatite is in a polycation-derivatized form at least during the contacting and washing. In some embodiments, the polycation is selected from the group consisting of polyethyleneimine, ethacridine, polyethanolamine, polylysine, polyarginine, and polyallylamine.

In some embodiments, the first wash buffer comprises sodium chloride, arginine, guanidine hydrochloride, urea, a surfactant, or a combination thereof. In some embodiments, the first wash buffer comprises sodium chloride and urea, sodium chloride and guanidine hydrochloride, or sodium chloride and arginine.

In some embodiments, the second viruicidal agent is sodium chloride or a chaotropic agent. In some embodiments, the chaotropic agent is arginine, guanidine, or urea.

In some embodiments, the first wash buffer comprises a sufficiently high conductivity and/or a sufficient amount of a chaotropic agent to elute the virucidal agent without substantially eluting the target biomolecule.

In some embodiments, the target biomolecule is labile at pH 4.

In some embodiments, the target biomolecule is a protein. In some embodiments, the protein is an antibody. In some embodiments, the antibody is an IgG or IgM antibody.

In some embodiments, the eluting comprises contacting the support with a solution comprising sodium phosphate.

The present invention also provides for methods of removing a positively-charged or neutral virucidal agent from a biomolecule preparation. In some embodiments, the method comprises, contacting a biomolecule preparation comprising a target biomolecule and a virucidal agent to an apatite support under conditions resulting in binding of the target biomolecule to the support such that the target biomolecule binds to the apatite and a majority of the virucidal agent flows past the support; and eluting the target biomolecule from the support such that the target biomolecule is substantially free of the virucidal agent.

In some embodiments, residual virucidal agent is associated with the target biomolecule on the support following the contacting step, and the method further comprises, between the contacting and eluting, washing the support with a first wash buffer, thereby eluting at least a majority of the residual virucidal agent while allowing substantially all of the protein target to remain bound to the support.

In some embodiments, the first wash buffer comprises sodium chloride, arginine, guanidine hydrochloride, urea, a surfactant, or a combination thereof. In some embodiments, the first wash buffer comprises sodium chloride and urea, sodium chloride and guanidine hydrochloride, or sodium chloride and arginine. In some embodiments, the first wash buffer comprises a sufficiently high conductivity and/or a sufficient amount of a chaotropic agent to elute the virucidal agent without substantially eluting the target biomolecule.

In some embodiments, the method further comprises, between the washing and eluting, contacting the support with a second wash buffer having a lower conductivity than the first wash buffer and no chaotropic agents.

In some embodiments, the apatite is hydroyxapatite or fluoroapatite.

In some embodiments, the target biomolecule is a protein. In some embodiments, the protein is an antibody. In some embodiments, the antibody is an IgG or IgM antibody.

In some embodiments, the virucidal agent is selected the group consisting of polyethyleneimine, ethacridine, chlorhexidine, benzalkonium chloride, tri(n-butyl)phosphate, and methylene blue.

In some embodiments, prior to or during the contacting, the apatite is contacted with a sufficient amount of a polycation to block negative charges of phosphate moieties within the apatite such that the virucidal agent does not substantially bind the apatite support.

In some embodiments, the polycation is selected from the group consisting of polyethyleneimine, polyethanolamine, polylysine, polyarginine, and polyallylamine.

In some embodiments, prior to or during the contacting, the apatite is contacted with a sufficient amount of a divalent or trivalent cation to block negative charges of phosphate moieties within the apatite such that the virucidal agent does not substantially bind the apatite support. In some embodiments, the divalent cation or trivalent cation is selected from the group consisting of calcium, iron, and zinc.

In some embodiments, the eluting comprises contacting the support with a solution comprising sodium phosphate.

In some embodiments, the conditions of the contacting, and optionally washing, do not comprise a detergent or hydrophobic molecule that disrupts an association of the virucidal agent and the target biomolecule.

The present invention also provides for an apatite chromatography support in contact with a target biomolecule and a positively-charged or neutral virucidal agent. In some embodiments, the target biomolecule is bound to the apatite chromatography support. In some embodiments, the apatite chromatography support is further in contact with a sufficient amount of a polycation to block negative charges of phosphate moieties within the apatite such that the virucidal agent does not substantially bind the apatite support. In some embodiments, the polycation is selected from polyethyleneimine, polyethanolamine, polylysine, polyarginine, and polyallylamine.

In some embodiments, the apatite is further in contact with a sufficient amount of a divalent or trivalent cation to block negative charges of phosphate moieties within the apatite such that the virucidal agent does not substantially bind the apatite support. In some embodiments, the divalent cation or trivalent cation is selected from the group consisting of calcium, iron, and zinc.

In some embodiments, the apatite is hydroxyapatite or fluoroapatite.

In some embodiments, the target biomolecule is a protein. In some embodiments, the protein is an antibody. In some embodiments, the antibody is an IgG or IgM antibody.

In some embodiments, the solid support or solution in contact to the solid support does not including a detergent or hydrophobic molecule that disrupts an association of the virucidal agent and the target biomolecule.

In some embodiments, the virucidal agent is selected the group consisting of polyethyleneimine, ethacridine, chlorhexidine, benzalkonium chloride, tri(n-butyl)phosphate, and methylene blue.

The present invention also provides for a polycation-derivatized apatite solid support. In some embodiments, a target biomolecule is bound to the apatite chromatography support. In some embodiments, the polycation is selected from polyethyleneimine, polyethanolamine, polylysine, polyarginine, and polyallylamine. In some embodiments, the apatite is hydroxyapatite or fluoroapatite. In some embodiments, the target biomolecule is a protein. In some embodiments, the protein is an antibody. In some embodiments, the antibody is an IgG or IgM antibody.

The present invention also provides for methods of purifying a biomolecule in a sample. In some embodiments, the method comprises contacting the sample to a polycation-derivatized apatite solid support; and purifying the target biomolecule. In some embodiments, the target biomolecule binds the solid support and is subsequently eluted, optionally following washing the support, thereby removing contaminants from the sample. In some embodiments, the target molecule flows past the solid support while at least some contaminants from the sample bind to the solid support. In some embodiments, the polycation is selected from polyethyleneimine, polyethanolamine, polylysine, polyarginine, and polyallylamine. In some embodiments, the apatite is hydroxyapatite or fluoroapatite. In some embodiments, the target biomolecule is a protein. In some embodiments, the protein is an antibody. In some embodiments, the antibody is an IgG or IgM antibody.

The present invention also provides for kits. In some embodiments, the kit comprises (i) an apatite chromatography support, and (ii) a positively-charged or neutral virucidal agent.

In some embodiments, the kit further comprises a polycation that can block negative charges of phosphate moieties within the apatite such that the virucidal agent does not substantially bind the apatite support. In some embodiments, the polycation is selected from polyethyleneimine, polyethanolamine, polylysine, polyarginine, and polyallylamine.

In some embodiments, the kit further comprises a divalent or trivalent cation that can block negative charges of phosphate moieties within the apatite such that the virucidal agent does not substantially bind the apatite support. In some embodiments, the divalent cation or trivalent cation is selected from the group consisting of calcium, iron, and zinc.

In some embodiments, the apatite is hydroxyapatite or fluoroapatite.

In some embodiments, the virucidal agent is selected the group consisting of polyethyleneimine, ethacridine, chlorhexidine, benzalkonium chloride, tri(n-butyl)phosphate, and methylene blue.

Also provided are methods of purifying a target biomolecule from a sample. In some embodiments, the method comprises contacting the sample to a polycation-derivatized apatite support; and collecting the target biomolecule following the contacting.

In some embodiments, the target biomolecule binds to the polycation-derivatized apatite support, and the method comprises subsequently eluting the target biomolecule from the support.

In some embodiments, the eluting comprises contacting the support with an increasing gradient of phosphate, borate, sulfate, monocarboxylates, and monocarboxylic zwitterions.

In some embodiments, at least one contaminant from the sample is washed from the support prior to elution of the target biomolecule.

In some embodiments, the target biomolecule is washed from the support while at least one contaminant from the sample remains bound to the support. In some embodiments, the target biomolecule is an antibody and the contaminant is DNA.

In some embodiments, the methods further comprise at least one wash after contacting the sample to the support and before elution of the target biomolecule. In some embodiments, the wash comprises washing the support comprising the bound target biomolecule with a solution comprising at least 0.5 M salt. In some embodiments, the wash comprises washing the support comprising the bound target biomolecule with a solution comprising a chaotropic agent. In some embodiments, the wash comprises washing the support comprising the bound target biomolecule with a solution comprising arginine.

In some embodiments, at least one contaminant in the sample binds to the polycation-derivatized apatite support and the target biomolecule flows through the support without substantially binding to the polycation-derivatized apatite support. In some embodiments, the contaminant is selected from the group consisting of DNA, virus, protein A and endotoxin.

In some embodiments, the biomolecule is a protein or a polynucleotide. In some embodiments, the biomolecule is a protein. In some embodiments, the biomolecule is an antibody. In some embodiments, the antibody comprises an IgG, IgM, or an antigen-binding fragment thereof.

In some embodiments, the polycation is selected from the group consisting of polyethyleneimine, polyethanolamine, polylysine, polyarginine, and polyallylamine, polyhistidine, polyornithine, polyethyleneimine, polydimethrine, polymethylacrylamidopropyltrimethylammonia, polydiallyldimethylammonia, polyvinylbenzyltrimethylammonia; polyvinylguanidine, poly(N-ethyl-4-vinylpyridine, DEAE-dextran, and DEAE-cellulose. In some embodiments, the apatite is hydroxyapatite or fluoroapatite.

Also provided is a polycation-derivatized apatite solid support, wherein the polycation is selected from polyethanolamine, polylysine, polyarginine, and polyallylamine, polyhistidine, polyornithine, polyethyleneimine, polydimethrine, polymethylacrylamidopropyltrimethylammonia, polydiallyldimethylammonia, polyvinylbenzyltrimethylammonia; polyvinylguanidine, poly(N-ethyl-4-vinylpyridine, DEAE-dextran, and DEAE-cellulose. In some embodiments, the apatite is hydroxyapatite or fluoroapatite.

Also provided are kits comprising a polycation-derivatized apatite solid support, wherein the polycation is selected from polyethanolamine, polylysine, polyarginine, and polyallylamine, polyhistidine, polyornithine, polyethyleneimine, polydimethrine, polymethylacrylamidopropyltrimethylammonia, polydiallyldimethylammonia, polyvinylbenzyltrimethylammonia; polyvinylguanidine, poly(N-ethyl-4-vinylpyridine, DEAE-dextran, and DEAE-cellulose. In some embodiments, the apatite is hydroxyapatite or fluoroapatite.

### BRIEF DESCRIPTION OF THE DRAWINGS

FIG. 1 is a chromatogram (profile 1) illustrating purification of IgM antibody that was treated with the virucidal agent PEI. The reagents and conditions were as follows: CHT™ type 1, 40 μm. 1 mL 1 mL/min. Equilibration and Wash 1 buffer: 50 mM Hepes, pH 7.0; Wash 2: 500 mM arginine, 2 M NaCl, 50 mM Hepes, pH 7.0; Wash 3: 50 mM Hepes, pH 7.0; Elution buffer: 10 250 mM sodium phosphate, pH 7.0; Clean with 500 mM phosphate. The CHT™ was converted to polycationic-derivatized CHT™ by injecting 5 mL 1% PEI-1300 in 50 mM Hepes, pH 7.0, as described in the Examples, and the CHT™ column was washed with equilibration buffer prior to the elution profile shown. The column was equilibrated with equilibration buffer, and 2 mL of the PEI-treated IgM supernatant was loaded onto the column. The column was washed to baseline with Wash 1 buffer; washed with Wash 2 buffer, washed with Wash 3 buffer, and eluted in a 10 column volumes linear gradient to elution buffer. The column was cleaned with 500 mM phosphate. Results were monitored with UV 254 and 280 nM profiles, a conductivity profile, and a pH profile.

FIG. 2 shows two control chromatograms illustrating the elution of IgM from a CHT™ I column under identical conditions as those for FIG. 1, except that the CHT™ was not treated with PEI (profile 2), or Wash 2 was omitted (profile 3).

FIG. 3 is a chromatogram (profile 1) illustrating purification of IgM antibody that was treated with the virucidal agent ethacridine, as described in the Examples. The DNA was removed from the IgM supernatant prior to treatment with ethacridine. The reagents and conditions were as follows: CHT™ II 40 treated with 0.00125% ethacridine, 1 mL 5×50, 1 mL/min. The buffers are as described above for FIG. 1, except that Wash 2 did not contain arginine. Results were monitored with UV 254, 280 and 365 nM profiles, a conductivity profile, and a pH profile.

FIG. 4 is a chromatogram (profile 2) illustrating purification of IgM antibody that was treated with the virucidal agent ethacridine, as described for FIG. 4, except that the CHT™ II was treated with 0.00625% ethacridine.

### DEFINITIONS

Terms are defined so that the invention may be understood more readily. Additional definitions are set forth throughout the detailed description.

“Apatite solid support” refers to a mineral of calcium and phosphate in a physical form suitable for the performance of chromatography. Examples include but are not limited to hydroxyapatite and fluorapatite. This definition is understood to include both the native and metal cation-derivatized forms of an apatite solid support.

“Hydroxyapatite” refers to a chromatography support comprising an insoluble hydroxylated mineral of calcium phosphate with the structural formula Ca₁₀(PO₄)₆(OH)₂. Its dominant modes of interaction are phosphoryl cation exchange and calcium metal affinity.

“Fluorapatite” refers to a chromatography support comprising an insoluble fluoridated mineral of calcium phosphate with the structural formula Ca₁₀(PO₄)₆F₂. Its dominant modes of interaction are phosphoryl cation exchange and calcium metal affinity.

“Ceramic” hydroxyapatite (CHT™) or “ceramic” fluorapatite (CFT™) refer to commercially-available (from Bio-Rad) forms of the respective minerals in which nanocrystals are agglomerated into particles and fused at high temperature to create stable ceramic microspheres suitable for chromatography applications. Commercial examples of ceramic hydroxyapatite include, but are not limited to, CHT™ Type I and CHT™ Type II. Commercial examples of fluorapatite include, but are not limited to, CFT™ Type I and CFT™ Type II. Unless specified, CHT™ and CFT™ refer to roughly spherical particles of any average diameter, including but not limited to about 10, 20, 40, and 80 microns. The choice of hydroxyapatite or fluorapatite, the type, and average particle diameter can be determined by the skilled artisan.

“Metal-derivatized apatite solid support” refers to an apatite solid support that has been treated with a divalent metal cation in the absence of phosphate buffer, to create a surface in which the negatively charged native apatite phosphate groups are neutralized by binding metal ions, and the metal ions are available to participate in coordination interactions with biomolecules such as proteins, polynucleotides, and viruses. One example includes apatites that are derivatized with calcium. This leaves a surface with the native calcium residues and the secondary calcium residues. Apatites derivatized with other metals would leave a surface of mixed metal character: the original calcium plus the derivatizing metal or metals.

“Cationic polymer-modified apatite support,” also referred to as a “polycation derivatized apatite support,” refers to an apatite solid support that has been treated with a positively charged polymer to create a surface in which the negatively charged native apatite phosphate groups are neutralized and excess positively charged groups on the polymer impart a net electropositive charge on the surface as a whole. Polycations, or “cationic polymers”, refer to molecules containing three or more positive charges, and in some embodiments, comprise 4 or more, 5 or more, 6 or more, 7 or more, 8 or more, or 9 or more positive charges within a single molecule. Polyethyleneimine is an example of a cationic polymer that can be used for this purpose. The polymer may range in size from a few hundred to more than 100,000 daltons. Other cationic polymers that may be used to product a similar effect include but are not limited to polylysine, polyarginine, and polyallylamine.

“Target molecule” or “target biomolecule” refers to a biomolecule, or molecule of biological origin, for purification according to the methods of the present invention. Target molecules include, but are not limited to, proteins, polynucleotides, viruses, and virus-like particles. Examples of proteins include but are not limited to antibodies, enzymes, growth regulators, clotting factors, and phosphoproteins. Examples of polynucleotides include DNA and RNA. Examples of viruses include enveloped and non-enveloped viruses.

“Antibody” refers to any immunoglobulin or composite form thereof. The term may include, but is not limited to, polyclonal or monoclonal antibodies of the classes IgA, IgD, IgE, IgG, and IgM, derived from human or other mammalian cell lines, including natural or genetically modified forms such as humanized, human, single-chain, chimeric, synthetic, recombinant, hybrid, mutated, grafted, and in vitro generated antibodies. “Antibody” may also include fusion proteins containing an immunoglobulin moiety. “Antibody” may also include antibody fragments such as Fab, F(ab′)2, Fv, scFv, Fd, dAb, Fc and other compositions, whether or not they retain antigen-binding function.

“Contaminant” or “complexed contaminant” refers to an unwanted constituent that is associated with a target molecule to be purified. The association may be either covalent or non-covalent without respect to the mechanism of association. Examples of contaminants include, but are not limited to, antiviral agents, proteins, nucleic acids, lipids, various cell culture media components and additives, metal ions, thioredoxins, sulfides, and endotoxins.

“Biomolecule preparation” and “biological sample” refer to any composition containing a target molecule of biological origin (a “biomolecule”) that is desired to be purified. In some embodiments, the target molecule to be purified is an antibody.

The term “detergent” refers to amphipathic, surface active, molecules with polar (water soluble) and nonpolar (hydrophobic) domains. Detergents bind strongly to hydrophobic molecules or molecular domains to confer water solubility. Examples of detergents are described in U.S. Pat. No. 5,883,256. In contrast to the use of detergents in U.S. Pat. No. 5,883,256, the present invention dissociates complexes of target molecules and virucidal agents by differential affinity to chromatography supports.

The term “polycation” includes molecules having a plurality of positive charges. For example, the term includes polyamines such as polyethanolamine, polylysine, polyarginine, and polyallylamine. Other exemplary polycations include, e.g., polyethyleneimine.

“Bind-elute mode” refers to an operational approach to chromatography in which the buffer conditions are established so that target molecules and, optionally undesired contaminants, bind to the ionic exchange ligand when the sample is applied to the ligand (which is optionally bound to a solid support). Fractionation of the target can be achieved subsequently by changing the conditions such that the target is eluted from the support. In some embodiments, contaminants remain bound following target elution. In some embodiments, contaminants either flow-through or are bound and eluted before elution of the target.

“Flow-through mode” refers to an operational approach to chromatography in which the buffer conditions are established so that the target molecule to be purified flows through the chromatography support comprising the ion exchange ligand, while at least some sample contaminants are selectively retained, thus achieving their removal.

### DETAILED DESCRIPTION OF THE INVENTION

### I. Introduction

Modification of apatite with polycations converts the apatite from a calcium affinity/cation exchange mixed mode support (referred to herein as “native form”) to a calcium affinity, anion exchange mixed mode support. With native form apatite, the cation exchange and calcium affinity mechanisms are sometimes antagonistic to one another. With DNA, for example, native apatite phosphoryl cation exchange groups repel the negatively-charged phosphate groups on DNA. DNA binding to the native apatite support is still achieved by calcium affinity, but binding is weakened. In contrast, modification of apatite supports with polycations blocks native phosphoryl cation exchange groups and replaces them with excess anion exchange groups in the form of the positive charges from the modifying polycation. The anion exchange functionality works cooperatively with calcium affinity to support enhanced retention of acidic contaminant molecules at conductivities ranging from zero to more than 200 mS/cm. This allows for use of the polycation-derivatized apatite support as an anion exchanger under high salt conditions and provides a unique ability for removing contaminants such as DNA, from IgG, IgM, or other antibody preparations. While the initial discovery was made in the context of antibody purification, it is believed that the methods can be adapted for use of purification of other biomolecules.

Natural and recombinant proteins produced in vivo or by in vitro cell culture carry an inherent threat of contamination by virus species that could be a direct threat to recipients of therapeutic proteins for treatment of a disease. Such products must be therefore treated to reduce or remove the threat of secondary virus infection. One common treatment is to expose virus to a low pH environment for a period of time, then restore the preparation to neutral pH. Another common treatment is to incubate the product containing preparation with virucidal agents. This kills virus but requires an additional step to remove the virucide after treatment. Because of the additional handling step, virucide treatment is mainly restricted to proteins that are not able to survive exposure to low pH.

There are at least two limitations with use of virucidal agents. One is that the virucidal agents may form complexes with the product that are sufficiently stable to survive simple removal processes. This allows low levels of residual virucide to persist in the treated preparation, which is a concern because virucides are inherently toxic. The other limitation is that not all virus species are inactivated adequately by the virucidal treatments commonly applied. For example, TNBP and surfactant treatments are known not to be effective for inactivation on protein-capsid retrovirus.

The present discovery addresses both of these limitations. Following binding the product to an apatite support, the product is washed with agents that dissociate residual first-stage virucide that may have formed stable complexes with product. Many of the agents that effectively dissociate those virucides are virucidal themselves and provide a second-stage inactivation step.

Apatite supports are particularly qualified for this application because of their ability to maintain strong protein binding even in the presence of high concentrations of neutral salts such as NaCl, in the presence of chaotropes such as urea, arginine, guanidine, and surfactants, all of which offer the combined abilities to inactivate virus and dissociate inactivating agents such as ethacridine etc from the protein product.

The discovery offers additional utility due to the ability of the methods described herein to enhance the removal of nonviral contaminants such as host proteins, DNA, and endotoxins. The enhancement in these cases arises from the ability of the second stage wash to dissociate complexes that may exist between the product and any of these contaminant classes.

Additionally, it has been surprisingly discovered that positively-charged or neutral virucidal agents can be removed from samples comprising target biomolecules using apatite chromatography support and a wash step that also functions as a separate anti-viral step, thereby providing a two-stage antiviral step. Many drug regulatory agencies require a number of different independent anti-viral steps when producing biologically-derived molecules for human or animal administration. By adapting one or more wash steps for removal of the virucidal agent to involve a high salt (and optionally also chaotropic agent) the wash step itself can function to both remove residual virucidal agent and to act as an antiviral step due to the anti-viral effects of the high salt (and chaotropic agent) step. Indeed, in view of apatite's affinity for certain viruses, the method can in some cases be considered to include three separate anti-viral mechanisms.

It has also been surprisingly discovered that an antibody can be purified from a virucidal agent by contacting the antibody and the virucidal agent with an apatite support, and eluting the bound target biomolecule from the support such that the antibody is substantially free of the virucidal agent. Conditions have been discovered in which the antibody binds to the support whereas a majority of the virucidal agent does not bind to the support and is eluted from the support. For example, in some cases, use of metal cation or polycation-derivatized apatites allow for this binding property. As noted above, as necessary or desired, residual virucidal agent that complexes with the target antibody can be removed with one or more wash step as detailed herein, which can be designed to have a separate anti-viral effect. For example, residual virucidal agent that remains associated with the target biomolecule bound to the support can be removed by washing the support with a buffer having a high conductivity and/or an amount of a chaotropic agent sufficient to elute the virucidal agent without eluting the biomolecule. In some embodiments, the bound biomolecule is then eluted by washing the support with a buffer having lower conductivity and no chaotropic agents. While the initial discovery was made in the context of an antibody, it is believed that the methods can be adapted for use of purification of other biomolecules.

### II. Methods

The methods of the present invention use apatite chromatography to purify a target molecule from a biological sample (a biomolecule preparation). Generally, the methods of the present invention involve contacting the sample comprising the target molecule to a polycation-derivatized apatite support and subsequently collecting the target molecule purified from one or more other components of the sample. In some embodiments, the method comprises contacting the sample comprising the target molecule to a polycation-derivatized apatite support, thereby non-covalently binding the target molecule to the apatite support; optionally washing the bound target molecule; and eluting the target molecule from the apatite support. Exemplary polycations include, but are not limited to, polyethyleneimine (PEI), polyethanolamine, polylysine, polyarginine, polyallylamine, polyhistidine, polyornithine, polyethyleneimine, polydimethrine, polymethylacrylamidopropyltrimethylammonia, polydiallyldimethylammonia, polyvinylbenzyltrimethylammonia, polyvinylguanidine, poly(N-ethyl-4-vinylpyridine, DEAE-dextran, and DEAE-cellulose.

In some aspects, the methods of the present invention involve contacting the sample comprising the target molecule, wherein the target molecule has been previously incubated with a positively-charged or neutral virucidal agent, to an apatite solid support, thereby non-covalently binding the target molecule to the apatite support; washing the bound target molecule where removal of residual complexed virucidal agent is required or desired, under conditions in which viruses are inactivated and the target molecule remains substantially bound to the apatite support; and then eluting the target molecule (substantially free of the virucidal agent) from the apatite support.

The methods can involve an initial incubation step in which the virucidal agent is incubated with the biomolecule preparation of a suitable time and under suitable conditions as known in the art to allow for the virucial agent to bind, disrupt, or otherwise interfere with viruses present in the preparation. After the incubation, the preparation containing the virucial agent can be contacted to the apatite chromatography support as described herein, either directly or after one or more initial purification steps. It will be appreciated that the sample incubated with the viruicidal agent can be added directly to the apatite support or can go through one or more purification or other steps prior to contact of the target molecule to the apatite support.

### Contacting Step

In some embodiments, prior to contacting the sample comprising the target biomolecule with the apatite support (e.g., apatite column), the chemical environment inside the column is equilibrated. Apatite supports in their native form generally comprise a large number of negatively charged phosphate (PO4⁻) moieties which significantly contribute to apatite's affinity for certain molecules. It has been discovered that treating the apatite support with a source of polycations, thereby blocking the negative charges of the phosphate moieties, allows for conditions in which target biomolecule nevertheless binds the polycation-treated apatite support, but other sample components (e.g., positively charged or neutral components) do not significantly bind the polycation-treated apatite support. It has also been discovered that treating the apatite support with a source of cations, thereby blocking the negative charges of the phosphate moieties, allows for conditions in which target biomolecule nevertheless binds the cation-treated apatite support, but the virucidal agent (which is typically positively charged or neutral) does not significantly bind the cation-treated apatite support. Accordingly, in some embodiments, the apatite support is preequilibrated with a solution comprising a cationic molecule that blocks the apatite phosphate moieties. This is accomplished, for example, by flowing an equilibration buffer comprising the cationic molecule through the column to establish the appropriate pH, conductivity, and concentration of salts.

In some embodiments, the cationic molecule used to block apatite phosphates is a divalent or trivalent cation, to generate a “cation-derivatized” apatite. Exemplary divalent or trivalent cations include, but are not limited to calcium, iron, or zinc. In embodiments in which apatite phosphates are blocked by divalent or trivalent cations, the equilibration buffer can include divalent or trivalent cation salts as appropriate, but generally will not include phosphate or other salts that remove (compete away) the divalent or trivalent cations from the apatite. The concentration of divalent or trivalent cation should be sufficient to block a sufficient amount of (e.g., essentially all) negative charges on the apatite surface such that the neutral or cationic virucidal agent does not significantly bind to the cation-derivatized apatite. In some embodiments the divalent or trivalent cation salts are at a concentration of about 2-5 mM. It may optionally include a buffering compound to confer adequate pH control. Buffering compounds may include but are not limited to MES, HEPES, BICINE, imidazole, and Tris. In some embodiments, the pH of the equilibration buffer for hydroxyapatite is from about pH 6.5 to pH 9.0. In some embodiments, the pH of the equilibration buffer for fluorapatite is from about pH 5.0 to 9.0.

In some embodiments, the apatite column is cation-derivatized with a solution comprising a metal cation salt at a concentration of about 2-10 mM, in the presence of one or more buffering compounds to confer adequate pH control. In some embodiments, the apatite column is calcium-derivatized, for example by applying an equilibration buffer comprising 5-10 mM calcium chloride in the presence of 20 mM HEPES and 20 mM MES and having a pH of about 7.

In some embodiments, the apatite column is derivatized (i.e., the apatite phosphates are blocked) by the presence of a polycation. Exemplary polycations include, but are not limited to, polyethyleneimine (PEI), polyethanolamine, polylysine, polyarginine, and polyallylamine. In embodiments in which apatite phosphates are blocked by polycations, the equilibration buffer can include polycations as appropriate, but generally will not include phosphate or other salts that remove the polycations from the apatite.

In some embodiments, the sample comprising the target biomolecule, interchangeably referred to herein as the biomolecule preparation, can also be equilibrated to conditions compatible with the column equilibration buffer before adding the sample to the column. This can include, for example, adjusting the pH, concentration of salts, and other compounds.

In some embodiments, the sample comprising the target molecule is contacted with a virucidal agent before contacting the sample with the column. In some embodiments, the sample comprising the target molecule is contacted with a virucidal agent selected from polyethyleneimine (PEI), ethacridine, chlorohexidine, benzalkonium chloride, tri(n-butyl)phosphate (TNBP), and methylene blue. The concentration of virucidal agent will depend on the specific agent used as well as the extent of viruses inhibition desired. In some embodiments, the sample is contacted with 0.01% PEI. In some embodiments, the sample is contacted with 0.001 to 0.010% ethacridine.

After the column and biomolecule preparation have been equilibrated, the biomolecule preparation can be contacted with the column under conditions that allow for the target molecule (which may be complexed with a residual amount of the virucidal agent) to bind to the cation-derivatized apatite. Generally, for example, protein binds to cation-derivatized apatites very strongly, and thus a variety of conditions can be used allowing for the target molecule to bind to the cation-derivatized apatite.

In some embodiments, the apatite solid support is derivatized with a metal cation or a polycation as described above, and the sample comprising a target molecule includes a positively charged virucidal agent. Thus, when the biological sample is contacted with the metal cation- or polycation-derivatized apatite, the positively charged virucidal agent is repelled by the positively charged apatite, thereby allowing the virucidal agent to pass through the column.

### Optional Washing Step

In some embodiments (e.g., where virucidal agents are to be removed), following binding of the target molecule to the apatite solid support, the bound target molecule can be washed with one or more agents that displace the complexed virucidal agent from the target molecule, or remove other contaminants (e.g., in the case where the target molecule is an antibody, DNA, endotoxin, residual host-cell proteins, and leached protein A are some undesirable contaminants), under conditions in which the target molecule remains substantially bound to the solid support. Without intending to limit the scope of the invention, it is believed that the agent(s) dissociate the target molecule from the virucidal agents by weakening the association (i.e., covalent interaction or non-covalent interaction) between them. The dissociating agent(s) act in combination with the apatite solid support, which itself functions to dissociate or displace virucidal agents from the target molecule.

In some embodiments, the dissociating agent also functions as a virucidal agent, resulting in a second stage of virus inactivation. For example, in some embodiments, the target biomolecule treated with a first virucidal agent is washed with a solution comprising a dissociating agent that is also a second virucidal agent. For example, in some embodiments, the wash step includes a wash step comprising an anti-viral agent selected from sodium chloride or a chaotropic agent (e.g., guanidine, arginine (see, e.g., Arakawa, et al., Biotechnol. J. 4(2): 174-178 (2009)), urea) or a combination thereof. Generally, a sufficient amount of these agents are used in the wash to achieve an anti-viral effect, e.g., to inactivate at least 50%, 90%, 95%, 99%, 99.9%, or more of virus present.

A variety of agents can be used to displace or dissociate the virucidal agents. Typically, the agent is a compound that does not substantially interfere with the binding of the target molecule to the apatite column (e.g., for a calcium-derivatized apatite column, the agent is one that lacks significant affinity for calcium).

In some embodiments, the dissociating agent is a chaotropic agent. Examples of chaotropic agents include, but are not limited to, compounds or molecules that destabilize hydrogen bonding and hydrophobic interactions, substances that increase the transfer of apolar groups to water, or disrupt the intermolecular forces between water molecules. Examples of chaotropic agents include guanidine hydrochloride, guanidine thyocyanate, lithium perchlorate, thiourea and urea. Notably, apatites, including hydroxyapatites, are highly tolerant of chaotropic agents, but the binding of a given protein is not always so. Proteins that bind by strong calcium affinity can tolerate high salt concentrations, in the absence of phosphate. Proteins with weak calcium affinity can be caused to tolerate high salt concentration if apatite is converted to its calcium-derivatized or another reation-derivatized form. Salt-tolerance permits protein binding to persist even, for example, in strong chaotropes such as 2 M guanidine.

In some embodiments, the dissociating agent is selected from the group consisting of arginine, urea, guanidine, sodium chloride, a salt lacking significant calcium affinity (e.g., NaCl, KCl, sodium acetate, potassium acetate, sodium perchlorate, potassium perchlorate, potassium isothiocyanate, guanidinium salts, amino acid salts, and thiocyanates), an organic solvent, a nonionic or zwitterionic surfactant, ethanol, and isopropanol.

In some embodiments, the agent is urea. Urea is also antiviral and is tolerated by all forms of hydroxyapatite (derivatized and underivatized) because urea is nonionic.

In some embodiments, the agent is sodium chloride. At sufficient concentrations, sodium chloride can also function as an antiviral agent.

In some embodiments, the agent is arginine. In some embodiments, the agent is guanidine or a salt thereof. Any conditions that permit use of guanidine will also tolerate arginine. Some users might prefer arginine because it has a milder effect while exploiting the same effect as guanidine through its guanido side group. For proteins that tolerate exposure to guanidine however, guanidine may be preferred, because guanidine is a more effective antiviral than arginine in some instances.

In some embodiments, the agent is a virucidal organic solvent. Exemplary organic solvents include, but are not limited to, ethylene glycols, propylene glycols, alcohols, DMSO, and DMF.

In some embodiments, the washing step comprises contacting the solid support binding the target molecule with one dissociating agent. In some embodiments, the washing step comprises contacting the solid support binding the target molecule with two, three, four, or more different dissociating agents. In some embodiments, the washing step comprises contacting the solid support binding the target molecule with a solution comprising the two or more different agents. As shown in the Examples section below, the use of a solution comprising at least two dissociating agents may increase the effectiveness of dissociating a complexed virucidal agent from a target molecule as compared to the use of each dissociating agent alone. In some embodiments, the two or more different dissociating agents comprise: arginine and sodium chloride, urea and sodium chloride; guanidine hydrochloride and sodium hydrochloride; urea, sodium chloride, and a reducing agent; a salt and an organic solvent; or a salt and a surfactant.

In some embodiments, the dissociating agent or agents are removed from the solid support prior to eluting the target molecule from the solid support. The agent or agents can be removed from the solid support, e.g., by washing the solid support with any suitable buffer (e.g., a “second wash buffer”) that does not elute the target biomolecule. For example, in some embodiments, the washing agents can be removed from the apatite support with a buffer comprising about 50 mM Hepes at about pH 7.

### Eluting Step

The target molecule can be eluted from the apatite solid support after the contacting and if it occurred, the washing step described above. In some embodiments, the apatite solid support from which the target molecule is eluted is converted from a metal cation (e.g., calcium)-derivatized form to a non-derivatized form after the washing step and during or prior to elution of the target molecule. In some embodiments, the apatite solid support from which the target molecule is eluted is converted from a polycation cation (e.g., PEI)-derivatized form to a non-derivatized form after the washing step and during or prior to elution of the target molecule. The metal cation-derivatized apatite solid support or polycation-derivatized apatite solid support can be converted to a non-derivatized form by contacting the apatite solid support, for example, with a phosphate buffer. In some embodiments, the derivatized apatite is converted to a non-derivatized condition by contacting the apatite solid support with a buffer comprising about 10 mM phosphate at about pH 7.

In some embodiments, the apatite solid support from which the target molecule is eluted remains in a metal cation (e.g., calcium)-derivatized form during the elution of the target molecule.

Elution conditions can comprise, for example, increasing the concentration of ion and/or buffer, thereby competing the target molecule from the support. For example, in some embodiments, the target molecule is eluted from a native form of apatite (i.e., converted back from the cation-derivatized form using phosphate) with a phosphate and/or sodium chloride gradient in which the buffer concentration is raised to, e.g., at least 250 mM, e.g., 250 mM-1.5 M, e.g., 500 mM-1.0 M. Optionally, the pH is maintained between pH 5.0-10.0, e.g., 5.5-8.5, e.g., between pH 6.5-7.5. Elution gradients can be linear or discontinuous.

In some embodiments, the target molecule is eluted with a linear gradient to about 250 mM sodium phosphate at a pH of between pH 6-8.

In some embodiments, at least 50%, 60%, 70%, 80%, 90%, 95%, or more of the target molecule bound to the solid support (in bind-elute mode) is eluted in the elution step.

In some embodiments, the target molecule that is eluted from the solid support is substantially free of contaminants. As used herein, “substantially free” means that the contaminants are 10% or less of the purified target molecule, e.g., less than 10%, 5%, 4%, 3%, 2%, 1%, 0.1%, 0.001%, or completely free of contaminants.

Whether complexed contaminants have been dissociated from the target molecule, and the extent to which complexed contaminants have been dissociated from the target molecule, can be determined by generating elution profiles for the chromatography run and looking at the pattern and/or size of peaks produced during the purification process. Additionally, when the target molecule or contaminant is DNA or protein, the removal of contaminants from the target molecule can be evaluated by measuring the A260 (absorbance at 260 nm; DNA) and/or A280 (absorbance at 280 nm; protein) profiles. In some embodiments, the removal of virucidal agents can be evaluated by measuring the A260, A280 and A 365 (absorbance at 365 nM; ethacridine) profiles. For example, elution profiles were generated for the Examples described herein.

The methods described herein can be performed at any scale (e.g., ranging from milligrams to kilograms of biological product per batc) and can be for any use, e.g., for research, diagnostic, therapeutic, or other applications.

### Optional Additional Steps

The present invention may be combined with other purification methods to achieve higher levels of purification. The chromatography step or steps may employ any method, including but not limited to size exclusion, affinity, anion exchange, cation exchange, protein A affinity, hydrophobic interaction, immobilized metal affinity chromatography, or mixed-mode chromatography. The precipitation step or steps may include salt or PEG precipitation, or precipitation with organic acids, organic bases, or other agents. Other fractionation steps may include but are not limited to crystallization, liquid:liquid partitioning, or membrane filtration.

The present invention may also be combined with additional virucidal treatments before or after the methods of the invention described herein.

### III. Target Biomolecules

The present invention provides methods of purifying a target biomolecule from a biological sample. In some embodiments, the target biomolecule in the biological sample is complexed with one or more virucidal agents.

Target biomolecules of the present invention include any biological molecule that may be purified using apatite chromatography. Examples of target biomolecules include, but are not limited to, proteins (e.g., antibodies, enzymes, growth regulators, clotting factors, and phosphoproteins), polynucleotides (e.g., DNA and RNA), viruses, and virus-like particles.

In some embodiments, the target molecule is an antibody or antibody fragment. In some embodiments, the antibody is an IgG, IgM, IgA, IgD, or IgE. Antibody preparations for use in the present invention can include unpurified or partially purified antibodies from natural, synthetic, or recombinant sources. Unpurified antibody preparations may come from various sources including, but not limited to, plasma, serum, ascites fluid, milk, plant extracts, bacterial lysates, yeast lysates, or conditioned cell culture media. Partially purified preparations may come from unpurified preparations that have been processed by at least one chromatography, precipitation, other fractionation step, or any combination of the foregoing.

The invention can be of particular interest in purification of proteins that are sensitive to low pH, which is one industry-standard method of virus reduction. Many recombinant proteins (including but not limited to clotting factors, including Factor VIII and von Willebrand Factor, and IgM antibodies) are highly labile and do not survive low pH treatment and thus are good candidates for the methods of the invention, and in particular those in which the wash step includes a second anti-viral agent. Moreover, some target proteins or other biomolecules ate too large to support reduction of non-enveloped viruses by filtration methods because the hydrodynamic radius of the virus is the same as the target biomolecule. These biomolecules are thus also particularly good candidates for use in the present methods.

### IV. Virucidal Agents

In some embodiments, methods of removing a virucidal agent from a biological sample are provided. In some embodiments, the methods are useful for dissociating one or more virucidal agents that are associated with a target molecule in order to enhance the purification of the target molecule. In some embodiments, the virucidal agent is positively charged. In some embodiments, the virucidal agent is neutral (not-charged). In some embodiments, the virucidal agent is polyethyleneimine (PEI), ethacridine, chlorhexidine, benzalkonium chloride, methylene blue, or tri(n-butyl)phosphate (TNBP).

### V. Kits

In another embodiment, the invention provides a kits for use in the methods described herein. A kit can optionally include written instructions or electronic instructions (e.g., on a CD-ROM or DVD) as well as packaging material. In some embodiments, the kits comprise an apatite chromatography support (e.g., a metal cation-derivatized or polycation-derivatized apatite) and a virucidal agent. Other reagents described herein in the context of the methods can also optionally be included in the kits.

### VI. Apatite Chromatography

The present invention provides for purifying a target molecule from a biological sample using an apatite solid support. Various apatite solid supports are available commercially, any of which can be used in the practice of this invention. These include but are not limited to hydroxyapatite and fluorapatite. Commercially available examples include but are not limited to ceramic hydroxyapatite (CHT™) or ceramic fluorapatite (CFT™). In some embodiments, the apatite solid support is a column.

In some embodiments, the apatite is selected from the group consisting of hydroxyapatite CHT™ Type I, 20 micron; hydroxyapatite CHT™ Type I, 40 micron; hydroxyapatite CHT™ Type I, 80 micron; hydroxyapatite CHT™ Type II, 20 micron; hydroxyapatite CHT™ Type II, 40 micron; hydroxyapatite CHT™ Type II, 80 micron; fluorapatite CFT™ Type I, 40 micron; and fluorapatite CFT™ Type II, 40 micron.

In some embodiments, CHT™ or CFT™ is packed in a column. In some embodiments, CHT™ or CFT™ is packed in a column of about 5 mm internal diameter and a height of about 50 mm, for evaluating the effects of various agents and combinations of agents on the dissociation of target molecule-virucidal agent complexes and elution characteristics of target molecules from a biomolecule preparation. In some embodiments, CHT™ or CFT™ is packed in a column of any dimensions required to support preparative applications. In some embodiments, column diameter may range from 1 cm to more than 1 meter, and column height may range from 5 cm to more than 30 cm depending on the requirements of a particular application. Appropriate column dimensions can be determined by the skilled artisan.

### Metal Cation-Derivatized Apatites

In some embodiments, the native hydroxyapatite and/or fluorapatite is converted to a metal cation-derivatized form by exposure to soluble metal cation in the absence of phosphate, thereby altering the selectivity of the apatite support. Examples of metal cations suitable for derivatization of native apatites include, but are not limited to, magnesium, zinc, iron, calcium, nickel, cobalt, manganese, copper, and chromium.

In some embodiments, the derivatized apatite is a calcium-derivatized apatite. Calcium derivatization largely eliminates apatite phosphate groups, replacing them with secondary calcium groups. Calcium derivatization increases the affinity of the apatite for phosphorylated molecules, thereby increasing the complex-dissociative potential of the support and increasing the effective purification of the target molecule of interest.

Methods of converting native apatite to a metal cation (e.g., calcium)-derivatized form are known in the art and are described, for example, in US 2009/0187005 and US 2009/0186396, each of which is incorporated by reference herein in its entirety. Briefly, an apatite solid support is equilibrated with a solution comprising a calcium salt at a concentration of about 2-5 mM, in the presence of one or more buffering compounds to confer adequate pH control. In some embodiments, the calcium salt is present at a concentration of about 1 mM to about 100 mM, about 1 mM to about 50 mM, about 1 mM to about 20 mM, or about 2 mM to about 10 mM. Buffering compounds may include but are not limited to MES, HEPES, BICINE, imidazole, and Tris. In some embodiments, the apatite is calcium-derivatized by applying to the apatite support a buffer comprising about 20 mM HEPES, about 20 mM MES, and about 5 mM calcium at about pH 7.

An apatite chromatography support of the present invention may be eluted in its metal cation (e.g., calcium)-derivatized form, or alternatively may be restored to its native (i.e., non-derivatized) form prior to elution. In some embodiments, metal cation-derivatized apatites are restored to their native forms by exposure to phosphate buffer, at which point they may be eluted by methods commonly applied for elution of native apatite supports. For example, calcium-derivatized apatite can be restored to native apatite upon washing with phosphate buffer. For some metal cation-derivatized apatites, the derivatization is only partially reversible or is irreversible. In some embodiments, the derivatized apatite (e.g., a calcium-derivatized apatite) is restored to its native condition by applying to the apatite support a buffer comprising about 10 mM phosphate.

### Polycation-Derivatized Apatites

In some embodiments, the native hydroxyapatite and/or fluorapatite is converted to a polycation-derivatized form by exposure to a soluble polycation in the absence of phosphate, thereby altering the selectivity of the apatite support. Example of polycations suitable for derivatization of native apatites include, but are not limited to, polyethyleneimine (PEI), and polyamines such as polyethanolamine, polylysine, polyarginine, and polyallylamine.

In some embodiments, the native hydroxyapatite is converted to a metal cation-derivatized apatite prior to being converted to a polycation-derviatized form. This conversion permits proteins that would otherwise be eluted by high salt washes from native or polycation-derivatized apatite to remain bound to the support.

Various methods of converting native apatite to a polycation cation-derivatized form can be used. Generally, polycation-derivatives are generated by contacting the apatite support with a solution containing a sufficient amount of a polycation, in the absence of phosphate, to displace the phosphate ions on the surface of the apatite. Y. Murakami, K. Sugo, M. Hirano, T. Okuyama, Talanta 85; 1298 (2011), for example, describes PEI-hydroxyapatite derivatives.

Derivitization of apatite supports can generally involve simply contacting the support with a solution containing a sufficient amount of the polycation at a pH in which the polycation is sufficiently cationic to bind to the apatite support. For example, in some embodiments, PEI or another polycation is titrated to a pH of about 6.5-7.0 and diluted, optionally ins a buffer such as 50 mM Hepes, to a concentration of 0.1%-2%. In some embodiments, the solid support is subsequently washed with a buffer (e.g., 50 mM Hepes, pH 7.0), followed by equilibration with 10 mM phosphate.

The concentration of polycation should be sufficient to block a sufficient amount of negative charges on the apatite phosphates such that cationic virucidal agent do not significantly bind to the polycation-derivatized apatite. Successful derivatization can be confirmed, for example, by applying a sample of DNA (e.g., 0.1 mg/mL salmon sperm DNA in 50 mM Hepes, pH 7.0) and comparing the phosphate concentration at which the DNA elutes in a phosphate gradient, to the eluting phosphate concentration in a native (not derivatized) apatite support column. DNA mostly elutes at about 250-300 mM phosphate from native CHT, but mostly not until 300-500 mM form polycation modified apatite. Cellular protein in typical biological samples, while containing some polycationic polypeptides, is not sufficient to block a sufficient amount of apatite phosphates for the purposes described herein.

The derivatization solution will generally include a buffering compound to confer adequate pH control. Ideally, the buffer will be positively charged or zwitterionic at the pH used (e.g., about pH 6-7.5, or, e.g., about 6.5-7.0) to avoid possible interactions of the buffer and the polycation. Buffering compounds may include but are not limited to MES, HEPES, histidine, histamine, and imidazole.

### EXAMPLES

The following examples are offered to illustrate, but not to limit the claimed invention.

The following examples describe removal of complexed virucidal agents from an IgM preparation. IgMs mostly elute from native-form apatites at high NaCl concentrations in the presence of low phosphate concentrations, thereby limiting, though not preventing, their purification on apatite supports prior to the discovery described herein. The use of calcium-derivatized or polycation-derivatized apatite permits IgM retention to be conserved at high NaCl concentrations, allowing the use of NaCl without limitation. Other salts without significant calcium affinity can likewise be used without limitation, potentially including but not limited to chaotropic salts such as guanidine, perchlorates, and thiocyanates. The salts can be used in combination with other dissociating agents as described above, for example, arginine and urea.

### Example 1

This example describes the removal of the polycation and virucidal agent PEI from a sample comprising IgM using the methods of the invention.

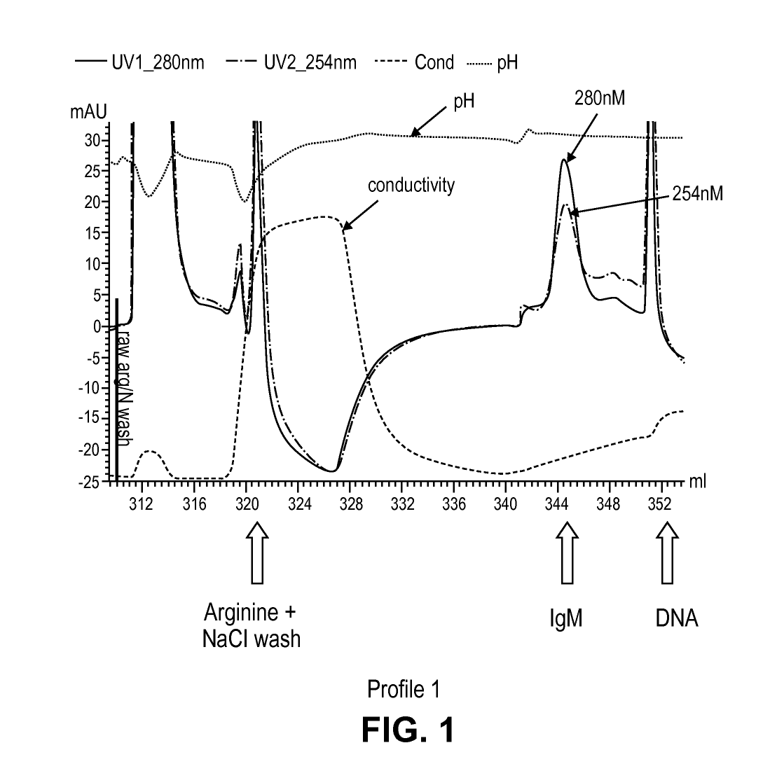

CHT™ type 140 micron was derivatized with PEI by injecting a 1% solution of PEI-1300 in 50 mM Hepes, pH 7. The derivatized CHT™ was equilibrated with 50 mM Hepes, pH 7. The sample contained IgM plus 0.01% PEI-1300. The sample was loaded onto the equilibrated, PEI-derivatized CHT™ column, and washed to baseline with equilibration buffer. The column was then washed with 500 mM arginine, 2 M NaCl, in 50 mM Hepes, pH 7. The column was then washed with equilibration buffer to remove the arginine and NaCl. The IgM was eluted using a 10 column volume linear gradient to 250 mM sodium phosphate, pH 7.0. The column was then cleaned with 500 mM phosphate, pH 7.0. The experiment was monitored at 254 and 280 nm UV. As shown in FIG. 1, the PEI was substantially removed from the column, as evidenced by a high 254 peak in the wash at 321 mL. The IgM was eluted at 344-346 mL, and the 254/280 ratio indicates the relative absence of PEI. The DNA eluted at 350 mL, as evidenced by the elevated 254 peak.

### Example 2

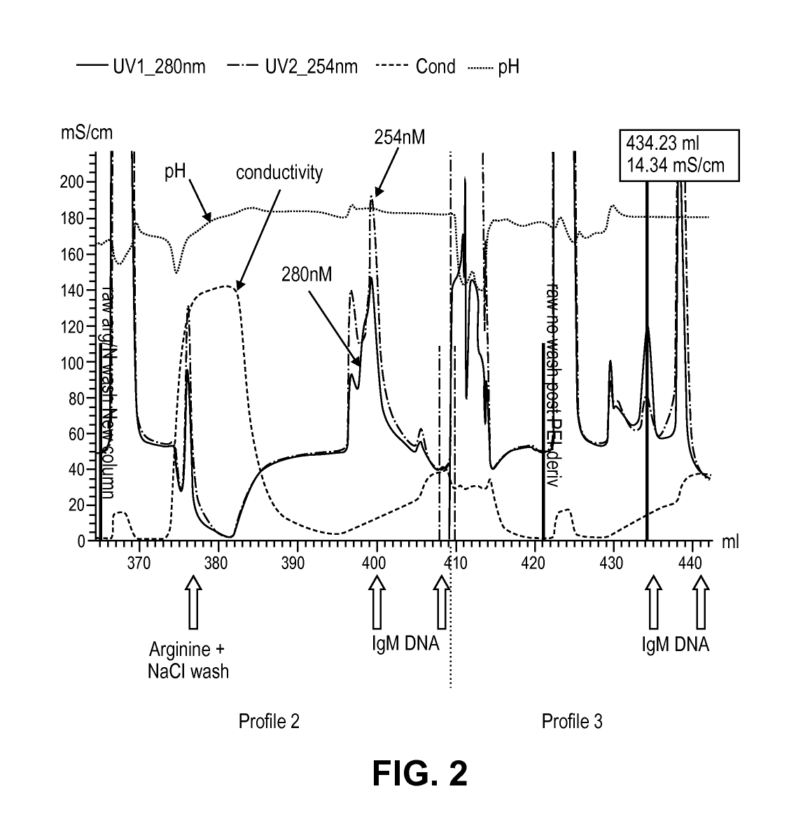

The experiment was repeated without making use of the invention, this time without treating the CHT™ with PEI. As shown in Profile 2 of FIG. 2, the 254 wash peak at 376 mL was much smaller, indicating the relatively little PEI was removed from the column during the arginine, NaCl wash. There was also a substantial contaminant peak at about 396 mL as indicated by the high 254 peak. The IgM eluted at about 399 mL, but contained high absorbance at 254, indicating that contaminants were present in the IgM fraction. The DNA peak at about 408 mL was substantially smaller than the DNA peak in Example 1, with the obvious corollary that DNA content of the eluted IgM must be higher.

### Example 3

The experiment was repeated as in Example 1 without making use of the invention, this time treating the CHT™ with PEI, but without the arginine/NaCl wash. As shown in Profile 3 of FIG. 2, the wash peak at about 429 mL showed a lower 245 absorbance peak, indicating that substantially less PEI was removed from the column. The IgM eluted at about 434 mL, and the DNA eluted at about 439 mL.

### Example 4

This example describes the removal of the virucidal agent ethacradine from a sample comprising IgM using the methods of the invention.

CHT™ type II 40 micron was derivatized with PEI as described in Example 1. The sample included IgM in which the DNA was removed that was treated with either 0.00125% (profile 1) or 0.00625% (profile 2) ethacridine. The PEI derivatized CHT™ was equilibrated and washed to baseline with equilibration buffer as described in Example 1. The column was washed with 2 M NaCl in 50 mM Hepes, pH 7, but without arginine. The column was then washed with equilibration buffer to remove the NaCl. The IgM was eluted and the column cleaned as described in Example 1. The experiments was monitored at 254, 280 and 365 nM UV. Ethacridine absorbs strongly at 365 nM.

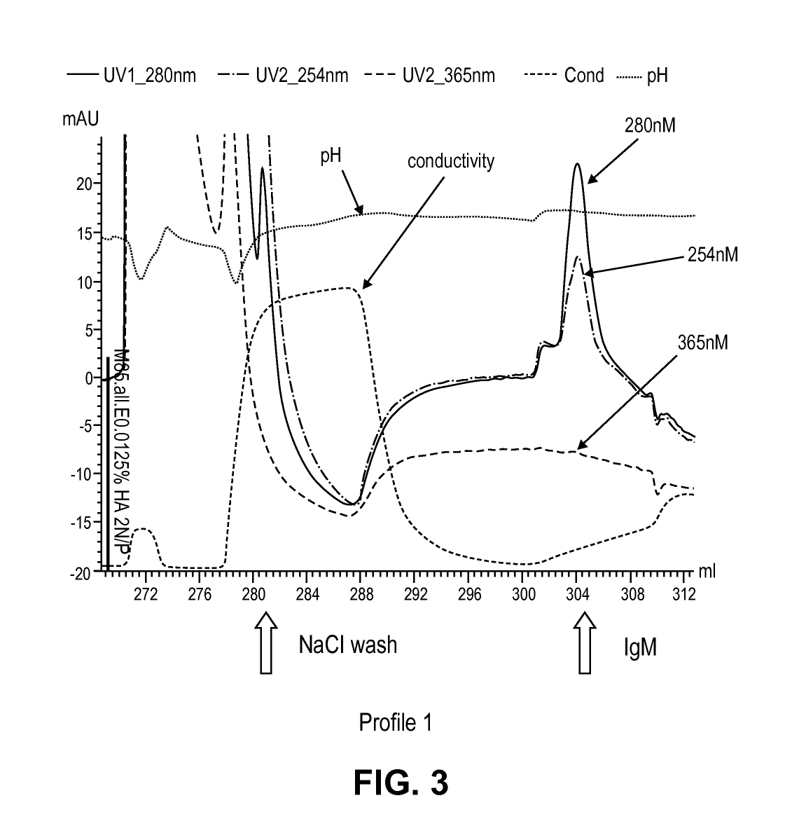

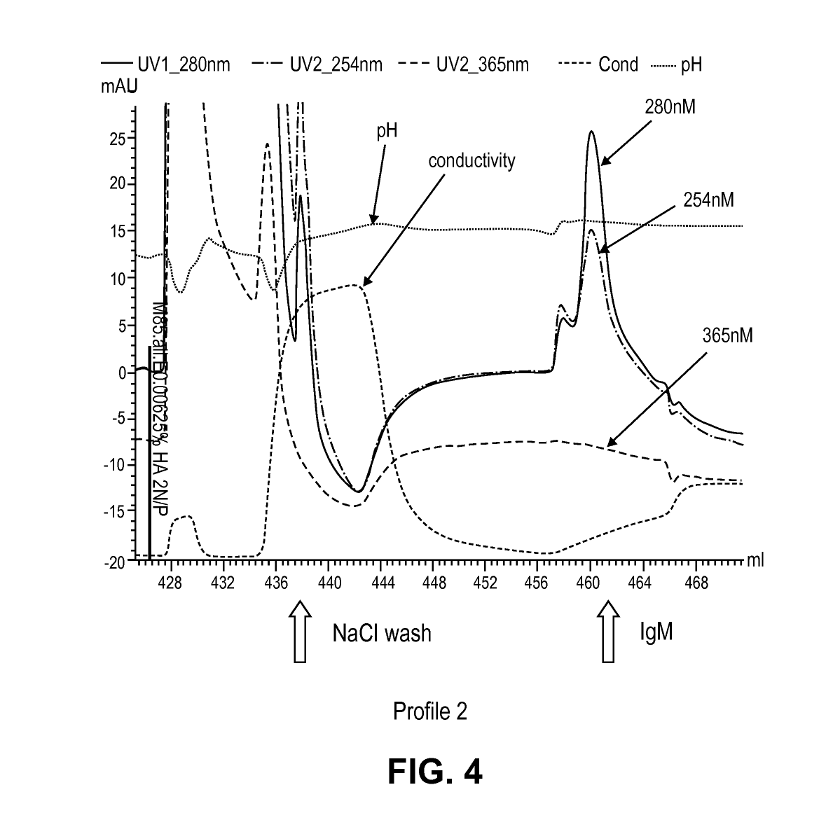

As shown in FIG. 3 (profile 1), most of the ethacridine failed to bind to the column, and the remainder was eliminated at the beginning of the NaCl wash, about 277-279 mL. The PEI eluted at the peak of the NaCl wash, as evidenced by the elevated 254 peak at about 280-281 mL. The 245/280 ratio and flat 365 absorbance indicates the relative absence of ethacridine and PEI beginning at 287 mL. The IgM eluted at 461 mL. As shown in FIG. 4 (profile 2), similar results were obtained using IgM treated with 0.00625% ethacridine.

The above examples demonstrate that the methods of the invention can remove virucidal agents and DNA contaminants that are complexed with antibodies, resulting in a relatively purified antibody preparation.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A two-stage viral inactivation method, comprising,
incubating a biological sample comprising a target molecule with a positively-charged or neutral virucidal agent under conditions to inactivate viruses in the sample, if present;
subsequently contacting the target molecule to an apatite support under conditions resulting in binding of the target biomolecule to the support such that the target biomolecule binds to the apatite and a majority of the virucidal agent flows past the support;
washing the support binding the target molecule with a first wash buffer, wherein the first wash buffer comprises at least a second virucidal agent, wherein the second virucidal agent is in sufficient concentration to inactivate viruses, if present, and to dissociate complexes of the positively-charged or neutral virucidal agent and the target molecule, thereby removing at least some residual virucidal agent, if present; and
eluting the target biomolecule from the support such that the target biomolecule is substantially free of the positively-charged or neutral virucidal agent.

2. The method of claim 1, wherein the positively-charged or neutral virucidal agent is selected the group consisting of polyethyleneimine, ethacridine, chlorhexidine, benzalkonium chloride, tri(n-butyl)phosphate, and methylene blue.

3. The method of claim 1, further comprising, between the washing and eluting, contacting the support with a second wash buffer.

4. The method of claim 3, wherein the second wash buffer has a lower conductivity than the first wash buffer and no chaotropic agents.

5. The method of claim 1, wherein the apatite is hydroyxapatite or fluoroapatite.

6. The method of claim 1, wherein the apatite is in a native form at least during the contacting and washing.

7. The method of claim 1, wherein the apatite is in a metal-derivatized form at least during the contacting and washing.

8. The method of claim 1, wherein the apatite is in a polycation-derivatized form at least during the contacting and washing.

9. The method of claim 8, wherein the polycation is selected from the group consisting of polyethyleneimine, polyethanolamine, polylysine, polyarginine, and polyallylamine.

10. The method of claim 1, wherein the second virucidal agent is sodium chloride or a chaotropic agent.

11. The method of claim 10, wherein the chaotropic agent is arginine, guanidine, or urea.

12. The method of claim 1, wherein the target biomolecule is labile at pH 4.

13. The method of claim 1, wherein the target biomolecule is a protein.

14. The method of claim 13, wherein the protein is an antibody.

15. The method of claim 1, wherein the eluting comprises contacting the support with a solution comprising sodium phosphate.

16. A method of removing a positively-charged or neutral virucidal agent from a biomolecule preparation, the method comprising,
contacting a biomolecule preparation comprising a target biomolecule and a virucidal agent to an apatite support under conditions resulting in binding of the target biomolecule to the support such that the target biomolecule binds to the apatite and a majority of the virucidal agent flows past the support; and
eluting the target biomolecule from the support such that the target biomolecule is substantially free of the virucidal agent.

17. The method of claim 16, wherein residual virucidal agent is associated with the target biomolecule on the support following the contacting step, and the method further comprises, between the contacting and eluting, washing the support with a first wash buffer, thereby eluting at least a majority of the residual virucidal agent while allowing substantially all of the protein target to remain bound to the support.

18. A kit comprising:
(i) an apatite chromatography support, and
(ii) a positively-charged or neutral virucidal agent.

19. The kit of claim 18, further comprising a polycation that can block negative charges of phosphate moieties within the apatite such that the virucidal agent does not substantially bind the apatite support.

20. The kit of claim 18, further comprising a divalent or trivalent cation that can block negative charges of phosphate moieties within the apatite such that the virucidal agent does not substantially bind the apatite support.
